(19) United States
(12) Patent Application Publication
(10) Pub. No.: US 2011/0027288 A1
(43) Pub. Date: Feb. 3, 2011

(54) HUMAN MONOCLONAL ANTIBODIES AGAINST AMYLOID BETA PROTEIN, AND THEIR USE AS THERAPEUTIC AGENTS

(76) Inventors: Moses Rodriguez, Rochester, MN (US); Larry R. Pease, Rochester, MN (US); Arthur E. Warrington, Rochester, MN (US); Nilufer Taner, Jacksonville, FL (US)

Correspondence Address: FISH & RICHARDSON P.C. (TC), PO BOX 1022, MINNEAPOLIS, MN, 55440-1022, US

(21) Appl. No.: 12/934,773

(22) PCT Filed: Mar. 24, 2009
(86) PCT No.: PCT/US09/38032

§ 371 Date: Sep. 27, 2010

Related U.S. Application Data
(60) Provisional application No. 61/039,615, filed on Mar. 26, 2008.

(51) Int. Cl. A61K 39/395; A61P 25/28

(52) U.S. Cl. 424/142.1

(57) ABSTRACT

Materials and methods for identifying human natural anti-Aβ autoantibodies, as well as materials and methods for using such antibodies to treat Alzheimer's disease.

### CROSS-REFERENCE TO RELATED APPLICATIONS

This application claims benefit of priority from U.S. Provisional Application Ser. No. 61/039,615, filed on Mar. 26, 2008.

### TECHNICAL FIELD

This document relates to materials and methods for treating or reducing development of Alzheimer's disease.

### BACKGROUND

Alzheimer's disease (AD) is the most common form of dementia in the elderly. Currently, no therapeutic strategies can prevent or reverse the progression of this disorder. AD is characterized by two pathological hallmarks: senile plaques composed of extracellular deposits of Aβ protein and neurofibrillary tangles, composed of the abnormally phosphorylated form of the microtubule associated protein, tau (τ). The identification of mutations in PS1 (presenilin-1), PS2, and amyloid precursor protein (APP) that lead to aggressive early-onset forms of AD provided the first clues into the pathogenesis of this disorder (Ertekin-Taner (2007)Neurol. Clin. 25:611-667). It has been established that 1) all of the early onset familial AD (EOFAD) mutations elevate Aβ in the brains, plasma and fibroblasts of mutation carriers and transgenic mice over-expressing the mutant protein, and 2) all EOFAD genes encode proteins that are members of the APP processing pathway (Citron et al. (1992) Nature 360:672-674; Cai et al. (1993) Science 259:514-516; Suzuki et al. (1994) Science 264:1336-1340; Borchelt et al. (1996) Neuron 17:1005-1013; Duff et al. (1996) Nature 383:710-713; Scheuner et al. (1996) Nat. Med. 2:864-870; Golde and Younkin (2001) Trends Mol. Med. 7:264-269; and Selkoe (2001) Physiol. Rev. 81:741-766). These findings have implicated Aβ as a central culprit in AD pathogenesis.

### SUMMARY

This document provides materials and methods for identifying one or more human anti-Aβ antibodies that may be useful to treat AD. The antibodies can be tested for their ability to lower the Aβ burden in a mouse model of AD. This document also provides anti-Aβ antibodies, compositions containing such antibodies, and methods for treating subjects having or at risk of developing AD by administering one or more anti-Aβ antibodies to the subject.

In one aspect, this document features a method for treating AD in a subject, comprising administering to the subject an anti-Aβ antibody, wherein the antibody is a natural human autoantibody from a subject having a monoclonal gammopathy. The antibody can be selected from the group consisting of Lym116, Lym128, Lym115, Lym118, Lym126, and Lym170. The administering can result in a decrease in the level of one or more previously observed AD symptoms. The symptoms can be selected from the group consisting of memory loss, difficulty performing familiar tasks, problems with language, disorientation to time and place, poor or decreased judgment, problems with abstract thinking, misplacing things, rapid changes in mood or behavior, changes in personality, and loss of initiative.

In another aspect, this document features a method for reducing development of AD symptoms in a subject, comprising administering to the subject an anti-Aβ antibody, wherein the antibody is a natural human autoantibody from a subject having a monoclonal gammopathy. The antibody can be selected from the group consisting of Lym116, Lym128, Lym115, Lym118, Lym126, and Lym170. The administering can result in a decrease in the level of one or more previously observed AD symptoms. The symptoms can be selected from the group consisting of memory loss, difficulty performing familiar tasks, problems with language, disorientation to time and place, poor or decreased judgment, problems with abstract thinking, misplacing things, rapid changes in mood or behavior, changes in personality, and loss of initiative.

In another aspect, this document features a composition comprising an anti-Aβ antibody and a pharmaceutically acceptable carrier, wherein the antibody is selected from the group consisting of Lym116, Lym128, Lym115, Lym118, Lym126, and Lym170.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DETAILED DESCRIPTION

This document provides materials and methods for treating AD. In particular, this document provides materials and methods for identifying one or more human natural anti-Aβ autoantibodies, as well as the anti-Aβ antibodies themselves, compositions containing the antibodies, and methods for treating subjects diagnosed as having or being at risk of developing AD.

### Antibodies

Antibodies that are present in the serum of healthy individuals in the absence of deliberate immunization with an antigen are referred to as “natural antibodies.” The majority of natural antibodies react with one or more self antigens and are referred to as “natural autoantibodies” (NAA). Autoreactive antibodies and B cells, as well as autoreactive T cells, are present in healthy individuals and in virtually all vertebrate species. Autoreactive antibody repertoires are predominantly selected early in development. Further, NAA have been cloned and sequenced, and appear to have genetic sequences very close to the host germline sequences. The pool of NAA in healthy subjects is critical for a variety of functions, including clearance of aging cells, antigen presentation, and anti-inflammatory activity.

While the NAA repertoire exists in all individuals, some individuals have a higher concentration, which is a condition known as monoclonal gammopathy. Monoclonal gammopathy indicates the presence of abnormal levels of a monoclonal immunoglobulin (Ig), also called an M-protein, in the blood. The protein is produced by plasma cells, which normally are found in the bone marrow and represent about one percent of all marrow cells. Plasma cells produce the antibodies that help the body fight infection. Abnormal proteins circulating in the blood are not rare; they occur in one percent of healthy people over the age of 50 and in three percent of people over the age of 70. In about 80 percent of cases, the abnormal protein does not cause any problems. Over time, however, about 20 percent of people experience an increase in the amount of abnormal protein in their blood, which can arise as a result of malignant transformation of plasma cells. This can develop into a more serious condition, including forms of cancer such as multiple myeloma. The reason for the monoclonal expansion of a single Ig-secreting plasma cell population in what appears to be a nonmalignant manner is unknown in most cases. Most cases involve IgG or IgA monoclonal cell populations, but about 15-20% involve IgM monoclonal cells.

In the screening methods provided herein, serum and/or plasma samples from humans with monoclonal gammopathies can be screened to identify anti-Aβ NAA. These methods can utilize efficient, high-throughput techniques to identify and test human anti-Aβ NAA that may be useful to treat AD. For example, serum samples from patients with monoclonal gammopathy can be subjected to screening assays for oligodendrocyte binding. Using such assays, monoclonal antibodies have been identified in monoclonal gammopathy patients with no history of neurological disease. Some of these antibodies have been shown to promote remyelination in a mouse model of multiple sclerosis (Warrington et al. (2000) Proc. Natl. Acad. Sci. USA 97:6820-6825). A similar approach may be useful to identify anti-Aβ autoantibodies for treating AD.

As used herein, the terms “antibody” and “antibodies” encompass intact molecules as well as fragments thereof that bind specifically to Aβ. An antibody can be of any immunoglobulin (Ig) class, including IgM, IgA, IgD, IgE, and IgG, and any subclass thereof. As used herein, an “epitope” is a portion of an antigenic molecule to which an antibody binds. Antigens can present more than one epitope at the same time. For polypeptide antigens, an epitope typically is about four to six amino acids in length. Two different immunoglobulins can have the same epitope specificity if they bind to the same epitope or set of epitopes. As used herein, “binds specifically to Aβ” means that a molecule binds preferentially to Aβ and does not display significant binding to other polypeptides (e.g., substantially less or no detectable binding to other polypeptides).

The terms “antibody” and “antibodies” include polyclonal antibodies, monoclonal antibodies, humanized or chimeric antibodies, single chain Fv antibody fragments, Fab fragments, and F(ab)₂ fragments. Polyclonal antibodies are heterogeneous populations of antibody molecules that are specific for a particular antigen, while monoclonal antibodies are homogeneous populations of antibodies to a particular epitope contained within an antigen.

Polyclonal antibodies are contained in the sera of immunized animals. Monoclonal antibodies also can be isolated from the serum of subjects (e.g., subjects having a monoclonal gammopathy, as described herein). Suitable methods for isolation include purification from serum using techniques that include, for example, chromatography. Monoclonal antibodies also can be prepared recombinantly, as described below, or by using standard hybridoma technology. For example, monoclonal antibodies can be obtained by any technique that provides for the production of antibody molecules by continuous cell lines in culture as described by Kohler et al. (1975) Nature 256:495-497, the human B-cell hybridoma technique of Kosbor et al. (1983) Immunol. Today 4:72, and Cote et al. (1983) Proc. Natl. Acad. Sci. USA 80:2026-2030, and the EBV-hybridoma technique of Cole et al., Monoclonal Antibodies and Cancer Therapy, Alan R. Liss, Inc. pp. 77-96 (1983). A hybridoma producing monoclonal antibodies can be cultivated in vitro or in vivo.

To isolate autoantibodies from human serum, clinical samples can be obtained (e.g., from a dysproteinemia clinic). Useful samples may be obtained from, for example, individuals having conditions characterized by a monoclonal IgM spike, including Waldenström macroglobulinemia, lymphoma, and monoclonal gammopathy of undetermined significance. Samples exhibiting an Ig clonal peak of greater than 20 mg/ml can be particularly useful for further evaluation. Antibodies can be isolated and characterized using standard methods. For example, antibodies can be precipitated from serum samples by centrifugation and purified using chromatography. Antibody (e.g., IgM) fractions can be pooled and analyzed by SDS-PAGE, and protein concentrations can be determined by reading absorbance at 280 nm.

Natural human autoantibodies that can be particularly useful include, for example, the human autoantibodies listed in Table 2 herein—Lym116, Lym128, Lym115, Lym118, Lym126, and Lym170.

Antibody fragments that have specific binding affinity for Aβ also can be generated. Such antibody fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced using standard techniques, such as those disclosed in U.S. Pat. No. 4,946,778. Such fragments can be rendered multivalent by, for example, biotinylation and cross-linking

### Nucleic Acids

Nucleic acids encoding human NAA that bind specifically to Aβ also are provided herein. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding the light and/or heavy chains of the antibodies provided herein.

An “isolated nucleic acid” refers to a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid.

The isolated nucleic acid molecules provided herein can be produced using standard techniques, including, without limitation, common molecular cloning and chemical nucleic acid synthesis techniques. For example, polymerase chain reaction (PCR) techniques can be used to obtain an isolated nucleic acid molecule encoding a human NAA that bind specifically to Aβ. Isolated nucleic acids also can be chemically synthesized, either as a single nucleic acid molecule (e.g., using automated DNA synthesis in the 3′ to 5′ direction using phosphoramidite technology) or as a series of polynucleotides. For example, one or more pairs of long polynucleotides (e.g., >100 nucleotides) can be synthesized that contain the desired sequence, with each pair containing a short segment of complementarity (e.g., about 15 nucleotides) such that a duplex is formed when the polynucleotide pair is annealed. DNA polymerase is used to extend the polynucleotides, resulting in a single, double-stranded nucleic acid molecule per polynucleotide pair.

This document also provides vectors containing nucleic acids such as those described above. As used herein, a “vector” is a replicon, such as a plasmid, phage, or cosmid, into which another DNA segment may be inserted so as to bring about the replication of the inserted segment. The vectors can be expression vectors. An “expression vector” is a vector that includes one or more expression control sequences, and an “expression control sequence” is a DNA sequence that controls and regulates the transcription and/or translation of another DNA sequence.

In the expression vectors provided herein, a nucleic acid (e.g., a nucleic acid encoding the light and/or heavy chains of a NAA) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors provided herein thus are useful to produce the antibodies provided herein.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

This document also provides host cells containing expression vectors. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Suitable methods for transforming and transfecting host cells are found in Sambrook et al., Molecular Cloning: A Laboratory Manual (2^{nd} edition), Cold Spring Harbor Laboratory, New York (1989), and reagents for transformation and/or transfection are commercially available (e.g., LIPOFECTIN® (Invitrogen/Life Technologies); FUGENE® (Roche, Indianapolis, Ind.); and SUPERFECT® (Qiagen, Valencia, Calif.)).

Once antibodies of interest are identified, immortalized sources thus can be generated to sustain these important reagents. As described in U.S. Pat. No. 7,052,694 and U.S. Patent Publication No. 2006/0099203, for example, a vector system can be developed and used to immortalize human antibodies such as the sHIgM12 (Lym12) and sHIgM22 (Lym22) antibodies, which were identified in the serum of a Waldenström macroglobulinemia patient. The amino acid sequence of the antibody can be determined from Fv fragments generated from the serum. Since malignant B cells circulate in the blood of Waldenström patients, cDNA encoding the heavy and light chain genes of the antibody present in highest serum concentrations can be successfully isolated. These cDNA sequences can be used to generate a genomic human IgM heavy chain gene encoding the variable region derived from the patient antibody and a cDNA-based light chain gene expressed under control of the cytomegalovirus (CMV) promoter. These antibody gene sequences then can be incorporated into a single vector along with a selectable dHfR gene expressed under the control of a SV40 promoter. In some embodiments, the cDNAs can be incorporated into a vector that has been modified for expression of IgM/Kappa antibodies by substituting the light chain constant region. The vector bearing the synthetic antibody genes can be introduced into hybridoma cells (e.g., F3B6 hybridoma cells) by electroporation. Methotrexate resistant cells can be selected and amplified by stepping up the amount of methotrexate in the culture medium. The recombinant antibodies can be tested to determine whether they display the functional properties identified for the antibodies isolated from patient serum. In addition, to ensure that recovered cDNAs truly represent the antibody of interest, the amino acid sequence of CDR3 regions of the serum antibodies can be determined by, for example, proteolytic digestion of the Fv fragments and conventional amino acid sequencing of the digestion products.

### Compositions

This document also provides compositions containing human anti-Aβ NAA as described herein. Such compositions are suitable for administration to subjects diagnosed as having AD. Methods for formulating and subsequently administering therapeutic compositions are well known to those skilled in the art. Dosages typically are dependent on the responsiveness of the subject to the molecule, with the course of treatment lasting from several days to several months, or until a suitable immune response is achieved. Persons of ordinary skill in the art routinely determine optimum dosages, dosing methodologies and repetition rates. Optimum dosages can vary depending on the relative potency of an antibody, and generally can be estimated based on the EC₅₀ found to be effective in in vitro and/or in vivo animal models. Dosage typically is from 0.01 μg to 100 g per kg of body weight (e.g., from 1 μg to 100 mg, from 10 μg to 10 mg, or from 50 μg to 500 μg per kg of body weight, or about 0.01 μg, 0.05 μg, 0.1 μg, 0.5 μg, 1 μg, 5 μg, 10 μg, 50 μg, 100 μg, or 500 μg per kg of body weight). Compositions containing the antibodies provided herein can be administered once or more daily, weekly, monthly, or even less often.

Antibodies can be admixed, encapsulated, conjugated or otherwise associated with other molecules, molecular structures, or mixtures of compounds such as, for example, liposomes, receptor targeted molecules, or oral, topical or other formulations for assisting in uptake, distribution and/or absorption.

Pharmaceutically acceptable carriers are pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water, saline solution, binding agents (e.g., polyvinylpyrrolidone or hydroxypropyl methylcellulose), fillers (e.g., lactose and other sugars, gelatin, or calcium sulfate), lubricants (e.g., starch, polyethylene glycol, or sodium acetate), disintegrates (e.g., starch or sodium starch glycolate), and wetting agents (e.g., sodium lauryl sulfate).

Pharmaceutical compositions containing the antibodies provided herein can be administered by a number of methods, depending upon whether local or systemic treatment is desired. Typically, the antibodies can be administered directly to the brain or the central nervous system. For administration to the central nervous system, for example, antibodies can be injected or infused into the cerebrospinal fluid. In some embodiments, an antibody can be administered with one or more agents capable of promoting penetration across the blood-brain barrier (BBB). Alternatively, administration can be, for example, parenteral (e.g., by subcutaneous, intrathecal, intraventricular, intramuscular, or intraperitoneal injection, or by intravenous drip), oral, topical (e.g., transdermal, sublingual, ophthalmic, or intranasal), or pulmonary (e.g., by inhalation or insufflation of powders or aerosols). Administration can be rapid (e.g., by injection) or can occur over a period of time (e.g., by slow infusion or administration of slow release formulations).

Compositions and formulations for parenteral administration can include sterile aqueous solutions (e.g., sterile physiological saline), which also can contain buffers, diluents and other suitable additives (e.g., penetration enhancers, carrier compounds and other pharmaceutically acceptable carriers). Compositions and formulations for oral administration can include, for example, powders or granules, suspensions or solutions in water or non-aqueous media, capsules, sachets, or tablets. Such compositions also can incorporate thickeners, flavoring agents, diluents, emulsifiers, dispersing aids, or binders. Formulations for topical administration can include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsion formulations are particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

The antibody compositions provided herein can encompass any pharmaceutically acceptable salts, esters, or salts of such esters, or any other compound which, upon administration to a subject, is capable of providing (directly or indirectly) the biologically active metabolite or residue thereof. Accordingly, for example, this document provides pharmaceutically acceptable salts of antibodies, prodrugs and pharmaceutically acceptable salts of such prodrugs, and other bioequivalents. The term “pharmaceutically acceptable salts” refers to physiologically and pharmaceutically acceptable salts of the antibodies useful in the methods provided herein (i.e., salts that retain the desired biological activity of the parent antibodies without imparting undesired toxicological effects). Examples of pharmaceutically acceptable salts include, but are not limited to, salts formed with cations (e.g., sodium, potassium, calcium, or polyamines such as spermine), acid addition salts formed with inorganic acids (e.g., hydrochloric acid, hydrobromic acid, sulfuric acid, phosphoric acid, or nitric acid), salts formed with organic acids (e.g., acetic acid, citric acid, oxalic acid, palmitic acid, or fumaric acid), and salts formed with elemental anions (e.g., bromine, iodine, or chlorine).

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the antibody components within the compositions provided herein.

Pharmaceutical formulations can be presented conveniently in unit dosage form, and can be prepared according to conventional techniques well known in the pharmaceutical industry. Such techniques include the step of bringing into association the active ingredients (i.e., the antibodies) with the desired pharmaceutical carrier(s). Typically, the formulations can be prepared by uniformly and intimately bringing the active ingredients into association with liquid carriers or finely divided solid carriers or both, and then, if necessary, shaping the product.

### Methods

This document provides methods for treating a subject diagnosed as having or being at risk for developing AD. The methods can include administering to a mammal (e.g., a human) one or more antibodies or a composition as described herein. As described above, one or more antibodies or a composition containing one or more antibodies can be administered by any suitable systemic or local method. Systemic methods of administration include, without limitation, oral, topical, or parenteral (e.g., intramuscular, intravenous, subcutaneous, transmucosal, or nasal) administration. Local methods of administration include, for example, direct injection into a particular site, such as injection into the brain or the cerebrospinal fluid. Thus, intracranial, intrathecal, or intraventricular administration can be used. In addition, the an antibody or a composition containing an antibody can be placed (e.g., injected) into the bloodstream after coupling the antibody to a carrier that will allow the antibody-carrier complex to cross the BBB. Such methods include those known to those skilled in the art, including those set forth, for example, in Banks et al. ((2007) Exp. Neurol. 206:248-256), and in PCT Publication No. WO 2006/103116. Strategies that can be useful for promoting transfer across membranes include, for example, increasing the hydrophobic nature of a molecule, introducing the molecule as a conjugate to a carrier such as a ligand targeted to a specific receptor, and the like.

In some embodiments, agents that increase the transfer of molecules through the BBB can be used (e.g., as described in U.S. Patent Publication No. 2006/0039859). Other methods for drug delivery through the BBB can include disruption of the BBB either by osmotic means (e.g., mannitol or leukotrienes) or biochemically by the use of vasoactive substances such as bradykinin. A BBB disrupting agent can be co-administered with an anti-Aβ antibody when the antibody is administered by intravascular injection, for example. Other strategies for crossing the BBB can include the use of endogenous transport systems, including carrier-mediated transporters such as glucose and amino acid carriers, receptor-mediated transcytosis for insulin or transferrin, and active efflux transporters such as p-glycoprotein. Active transport moieties also can be conjugated to an antibody to facilitate transport across the epithelial wall of the blood vessel. Alternatively, delivery across the BBB can occur by intrathecal delivery directly to the cranium (e.g., through an Ommaya reservoir).

The methods provided herein can be used to treat AD, or to delay or inhibit development of AD. For example, administration of a human anti-Aβ NAA can result in a decreased level of previously observed AD symptoms, or can result in decreased development of new symptoms. Symptoms of AD can include, without limitation, memory loss, difficulty performing familiar tasks, problems with language, disorientation to time and place, poor or decreased judgment, problems with abstract thinking, misplacing things, rapid changes in mood or behavior, changes in personality, and loss of initiative.

Methods for treating or inhibiting development of AD can include administering to a subject (e.g., a mammalian subject such as a dog, cat, rabbit, rodent, or human) an effective amount of a human anti-Aβ NAA, or an effective amount of a composition containing such an antibody. As used herein, the term “effective amount” is an amount of an antibody or composition that is sufficient to reduce or inhibit development of at least one AD symptom in a subject. For example, an “effective amount” of an antibody can be an amount that reduces or inhibits development of an AD symptom in a treated subject by at least 10% as compared to the level of the symptom in the subject prior to administration of the antibody. In some embodiments, methods can include administering to a mammal an amount of an antibody or composition that is sufficient to reduce or inhibit development of AD symptoms by at least 50%. It is noted that in some embodiments, the methods provided herein also can include steps for identifying a subject as having or being at risk for AD, and/or monitoring treated subjects for a reduction in symptoms of AD.

### Articles of Manufacture

This document also provides articles of manufacture that can include the antibodies and/or compositions provided herein. The antibodies and/or compositions can be combined with packaging material and sold as kits for treating AD in an individual. Components and methods for producing articles of manufacture are well known. Articles of manufacture may combine one or more of the antibodies identified as described herein, or can contain a composition that includes an antibody as provided herein. An article of manufacture also may include, for example, buffers or other reagents for treating AD. Instructions describing how the antibodies and compositions are effective for treating AD can be included in such kits.

The invention will be further described in the following examples, which do not limit the scope of the invention described in the claims.

### EXAMPLES

### Example 1

### Initial Screen of 76 Samples from Patients with Monoclonal Gammopathies

High-throughput ELISA screening to identify Aβ-binding activity in human samples: Human serum samples were obtained from the dysproteinemia clinic at Mayo Clinic Rochester. Samples were chosen based on the criterion of having an immunoglobulin (Ig) clonal peak >20 mg/ml. Sera were from patients with monoclonal IgG or IgM gammopathies due to a variety of conditions, including Waldenström macroglobulinemia, multiple myeloma, lymphoma, and monoclonal gammopathy of undetermined significance. The human monoclonal Igs were isolated from sera as described previously (Warrington et al., supra). Aβ₁₋₄₀ and Aβ₁₋₄₂ coated 96-well ELISA plates were obtained from Dr. Chris Eckman at Mayo Clinic Jacksonville. The wells on these plates (Nunc-Immuno™ plates with MAXISORP™ surface) were loaded with 100 μl of either peptide (obtained from BACHEM™) at a concentration of 1385.4 nM and incubated overnight at 4° C. The following day, the Aβ solutions were removed from the plates and a blocking agent (BlockAce) was added to each well, followed by another overnight incubation at 4° C. The Aβ-incubation step was omitted for negative control plates that were used to detect non-specific plate-binding.

Purified monoclonal antibody samples were diluted to a concentration of 10 μg/ml in a 1×PBS+1% bovine serum albumin (BSA)+0.1% Tween solution. After addition of the samples, the plates were incubated at room temperature for 1.5 hours, followed by a washing step. Horse radish peroxidase- (HRP-) tagged sheep anti-human Ig was added to each well, followed by a second incubation period of 1.5 hour. The plates were developed and the absorbance of each well at 450 nm was measured using a plate reader.

Each sample was added in duplicate to three different plates coated with Aβ₁₋₄₂, Aβ₁₋₄₀, or BlockAce alone. Each experiment was repeated twice, and each plate had a number of negative control wells with only buffer added. The optical density (OD) readings of the duplicates were averaged and normalized by subtracting the averaged OD readings of all negative control wells. The averaged and normalized OD readings for each sample from the two experiments were averaged again and used in the final analyses. The final OD for each sample on the Aβ plate was divided by that for the BlockAce plate to obtain a ratio. Any sample with a ratio of 1.5 or above was considered to be a “hit” for this initial screen. Based on these criteria, samples from the initial set were identified to be hits for Aβ₁₋₄₂ binding and/or for Aβ₁₋₄₀ binding. Characteristics of the six samples with the highest ratios are summarized in Table 1.

All of the hits in the initial ELISA screen were IgM antibodies. This was consistent with previous findings using an oligodendrocyte screen (Warrington et al., supra). None of the patients whose samples bound to the Aβ plates had AD, another finding consistent with the notion that autoantibodies naturally occurring in healthy subjects represent a primordial pool with physiologic function as opposed to a “reactive” pool.

The secondary immunohistochemical screen of the ELISA hits: The human antibody samples are submitted to a secondary immunohistochemistry screen. To determine the specificity of these antibodies for the Aβ pathology in AD, each sample is incubated with brain tissue from AD patients that are prepared as fresh-frozen or paraffin-embedded sections. Secondary anti-human Igs are added and stained to visualize the binding pattern of these antibodies (e.g., to determine whether they display strong, specific and/or widespread binding to Aβ plaques, binding to cored plaques, and/or binding to neurofibrillary tangles. It is noted that binding (or lack thereof) of anti-Aβ antibodies to Aβ plaques in vitro may not necessarily be a direct predictor for whether the antibody will be efficient in reducing plaque burden. Since all of the murine anti-Aβ antibodies with substantial effects on Aβ clearance are strong binders of Aβ plaques (Bard et al. (2000) Nat. Med. 6:916-919), antibodies that “fail” this secondary screen are not further pursued.

TABLE 1
Characteristics of samples that bound
Aβ plates in the ELISA screen

| | Aβ:negative | Aβ:negative | | | |
|---|---|---|---|---|---|
| Sample | control ratio^{a} | control ratio^{a} | | | |
| code | Aβ type 40 | Aβ type 42 | Age^{d} | Gender | Diagnosis |
| Lym116 | 12.57 | 12.57 | 79 | F | WM^{b} |
| Lym128 | 3.99 | 7.04 | 69 | M | WM |
| Lym115 | 6.18 | 6.67 | 80 | F | Cold |
| | | | | | agglutination |
| | | | | | disease |
| Lym118 | 2.95 | 2.38 | 77 | M | MGUS^{c} |
| Lym126 | 4.29 | 3.62 | 81 | F | WM |
| Lym170 | 3.23 | 4.26 | 67 | F | WM |

^{a}Averaged normalized OD ratio for samples on the Aβ plate:negative control plate;
^{b}Waldenström macroglobulinemia;
^{c}Monoclonal gammopathy of undetermined significance;
^{d}At date of collection.

### Example 2

### Further Screens

ELISA screen: This initial screen is essentially performed as described above, but instead of testing purified antibody samples, direct testing of the serum samples is done to maintain a sufficiently high-throughput. As the amount of monoclonal antibody in the serum/plasma is recorded for each sample in a central database at the Mayo Clinic, all samples are diluted to the same concentration for the ELISAs. Given an estimated collection rate of ˜200 samples/year and an ongoing collection for >30 years, there are at least several thousand monoclonal gammopathy samples from different patients at the Mayo Clinic dysproteinemia laboratory. The clinical details associated with the patients, along with sample details are part of the records at the Mayo Clinic. Samples are regularly obtained from this unique repository for the anti-Aβ autoantibody identification project.

Immunohistochemistry screen: Samples with a ratio≧1.5 for Aβ plate binding: BlockAce plate binding is submitted to a secondary screen that involves staining of fresh frozen and paraffin-embedded AD brain sections. Monoclonal antibodies from the “candidate” samples are first purified as described previously (Warrington et al., supra). Immunostaining of brain sections is performed using an immunoperoxidase technique. Given that anti-Aβ antibodies shown to have Aβ-reducing effects in previous studies strongly bind Aβ plaques (Bard et al., supra), those antibodies with weak, no, or non-specific binding are eliminated.

Testing of anti-Aβ human antibodies in the Tg2576 mouse model of AD: Antibodies that pass both the ELISA and immunohistochemistry screens are tested in the Tg2576 mouse model of AD (Hsiao et al. (1996) Science 274:99-102). Based on the pilot experiments presented above, a hit rate of 3-5% is expected at the ELISA screen. A lower rate is expected for the immunohistochemistry screen. If a hit rate of 0.5% is assumed, at the end of screening 2000 serum samples one would expect to have ten antibodies to evaluate in vivo. For the purpose of these studies, however, the aim is to identify two antibodies that bind Aβ in vitro and lower Aβ levels in vivo. If two hits are identified that are effective in the Tg2576 model before screening all of the serum samples in the repository, screening is stopped and efforts are concentrated on sequencing the two hits.

Age-dependent changes in the brain, CSF, and plasma Aβ of Tg mice have been characterized (Kawarabayashi et al. (2001) J. Neurosci. 21:372-381), and a detailed time-course of these changes has been tracked. These experiments demonstrated that Tg2576 mice start depositing insoluble forms of Aβ at about seven months of age. By eight to nine months of age, this insoluble form is observed in the brain of every Tg2576 mouse, and the increase is exponential between 6-12 months of age. Immunocytochemically cored plaques appeared at seven to eight months of age and increased between seven and ten months of age, with a few cores present at each section by ten months. This detailed knowledge on the temporal accumulation of Aβ is utilized in designing treatment experiments. The previous studies on AD immunotherapy in Tg mice utilized a range of treatment protocols that were initiated and terminated at various time-points. The two main approaches are 1) administration of treatment before the onset of Aβ pathology, and 2) administration of treatment after development of significant Aβ pathology.

For the present studies, the aim is to choose time points that allow demonstration of a significant prevention in development of Aβ pathology (as in approach 1), as well as determination of reversal if present (as in approach 2). Given the existence of biochemically demonstrable Aβ pathology at nine months that goes through an exponential increase until twelve months of age, treatment is started at 9 months of age and pathology is assessed both biochemically and by immunocytochemistry at 11 months. Because of the exponential rise in the amount of insoluble Aβ between 9 and 11 months that results in a ˜10 fold increase, the effects of a treatment that halts the progression should be readily detected.

Assuming a non-conservative coefficient of variation estimate at 25%, there is >80% power to detect a modest effect size of 30% decrease in Aβ accumulation at α=0.05, by using 10 animals each in the treatment and control groups. Ten Tg2576 mice are injected intraperitoneally (i.p.) with a single 0.5 mg dose of anti-Aβ antibody. Given that the serum samples have high concentrations of monoclonal antibody spikes (2-10 mg/ml) in large volumes (>10 ml), there is enough antibody to perform the initial mouse treatment experiment as well as additional studies.

A group of ten Tg2576 mice also is injected with a negative control human antibody with no Aβ binding either on the Aβ plate or brain sections. Two such antibodies have been tested in an in vivo pilot study. One of these is a remyelinating antibody from a previous oligodendrocyte screen, and the other is a hit for the Aβ screen that did not bind the Aβ plaques on the brain sections. Based on the results of this pilot experiment, one of these antibodies could be used as a negative control in future in vivo studies. A group of PBS-injected animals also is used for each treatment cycle.

Mice are sacrificed by decapitation. After removal, the brain is cut into two hemibrains by a coronal section. One of the hemibrains is frozen in liquid nitrogen for future Aβ measurements, and the other is placed in paraformaldehyde for immunocytochemistry. Brain Aβ measurements and immunohistochemistry are performed as described previously (Kawarabayashi et al., supra). The amount of Aβ deposition in the treatment vs. control groups is determined using non-parametric Mann-Whitney tests. Any anti-Aβ antibody with an effect towards reduction of Aβ in the Tg2576 mouse model of AD is analyzed further by additional in vivo studies. Antibody(ies) with a consistent lowering effect on brain Aβ are sequenced to allow for generation of a recombinant molecule for mass production. Such procedures are described, for example, in U.S. Pat. No. 7,052,694 and U.S. Patent Publication No. 2006/0099203.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating Alzheimer's disease (AD) in a subject, comprising administering to said subject an anti-Aβ antibody, wherein said antibody is a natural human autoantibody from a subject having a monoclonal gammopathy.

2. The method of claim 1, wherein said antibody is selected from the group consisting of Lym116, Lym128, Lym115, Lym118, Lym126, and Lym170.

3. The method of claim 1, wherein said administering results in a decrease in the level of one or more previously observed AD symptoms.

4. The method of claim 3, wherein said symptoms are selected from the group consisting of memory loss, difficulty performing familiar tasks, problems with language, disorientation to time and place, poor or decreased judgment, problems with abstract thinking, misplacing things, rapid changes in mood or behavior, changes in personality, and loss of initiative.

5. A method for reducing development of AD symptoms in a subject, comprising administering to said subject an anti-Aβ antibody, wherein said antibody is a natural human autoantibody from a subject having a monoclonal gammopathy.

6. The method of claim 5, wherein said antibody is selected from the group consisting of Lym116, Lym128, Lym115, Lym118, Lym126, and Lym170.

7. The method of claim 5, wherein said administering results in a decrease in the development of one or more AD symptoms.

8. The method of claim 7, wherein said symptoms are selected from the group consisting of memory loss, difficulty performing familiar tasks, problems with language, disorientation to time and place, poor or decreased judgment, problems with abstract thinking, misplacing things, rapid changes in mood or behavior, changes in personality, and loss of initiative.

9. A composition comprising an anti-Aβ antibody and a pharmaceutically acceptable carrier, wherein said antibody is selected from the group consisting of Lym116, Lym128, Lym115, Lym118, Lym126, and Lym170.
